(19) United States
(12) Patent Application Publication
(10) Pub. No.: US 2024/0423930 A1
(43) Pub. Date: Dec. 26, 2024

(54) PHARMACEUTICAL COMPOSITION AND METHOD FOR TREATING CARDIOVASCULAR DISEASES

(71) Applicant: David BEIHL, Saluda, SC (US)

(72) Inventor: David BEIHL, Saluda, SC (US)

(21) Appl. No.: 18/827,695

(22) Filed: Sep. 7, 2024

Related U.S. Application Data
(63) Continuation-in-part of application No. 17/516,877, filed on Nov. 2, 2021.
(60) Provisional application No. 63/108,704, filed on Nov. 2, 2020.

(51) Int. Cl. A61K 31/137; A61K 31/135; A61K 31/5377; A61K 33/00; A61K 47/69

(52) U.S. Cl. CPC A61K 31/137; A61K 31/135; A61K 31/5377; A61K 33/00; A61K 47/6951

(57) ABSTRACT

A pharmaceutical composition having a respiratory stimulant and a second compound including one or more naturally-occurring non-radioactive isotopes of a noble gas. The pharmaceutical composition combines with a proprotein convertase to form a therapeutically effective pharmaceutical enzyme inhibitor. A method of preventing or treating a cardiovascular disease characterized by TGF-beta signaling mechanism. A pharmaceutical composition including a respiratory stimulant and a naturally-occurring non-radioactive isotope of a noble gas is administered to the patient. The noble gas combines with proprotein convertase present in the patient's body to inhibit proprotein convertase. The proprotein convertase inhibition effected by the pharmaceutical composition decreases levels of TGF-beta production to prevent or treat the cardiovascular disease in the patient.

### CROSS REFERENCE TO RELATED APPLICATIONS

This application is a continuation-in-part of co-pending U.S. patent application Ser. No. 17/516,877 entitled Pharmaceutical Composition and Method for Treating Cardiovascular Diseases filed Nov. 2, 2021, which claims the benefit under 35 U.S.C. § 119(e) from U.S. Provisional Patent Application No. 63/108,704 entitled A Pharmaceutical Composition and Method for Treating Cardiovascular Diseases filed on Nov. 2, 2020.

### BACKGROUND OF THE INVENTION

### 1. Field of the Invention

The invention relates to a pharmaceutical composition and method for treating cardiovascular diseases.

### 2. The Prior Art

The proprotein convertases are a group of enzymes, found in humans and other organisms, whose primary known function is to activate other proteins. In humans, there are presently nine recognized proprotein convertases. Each enzyme of this group operates by proteolytic cleavage of another protein's inactive form (the proprotein), after which the protein's activity is changed (usually increased). In some cases, the portion which is removed is also known to have biologic function. The proprotein convertases are generally felt to have overlapping, related but distinct functions from each other.

Some cardiovascular diseases are facilitated by the function of proteins activated by proprotein convertases. Medications that inhibit certain proprotein convertases might prevent or reverse these diseases. However, the most commonly used proprotein convertase inhibitors have a narrow therapeutic index, which means the dose that inhibits the enzyme is similar to the dose that causes toxicity in the patient. One example of this is dec-RVKR-cmk (where RVKR is SEQ ID NO:1, and the example is in the form X-SEQ ID NO:1-Y, where X is decanoyl and Y is chloromethylketone). Others are complex biological products that can be difficult to synthesize, expensive to produce, or too large in size to enter cells or be absorbed effectively by the body or delivered to the cardiovascular system. In addition, the efficacy and toxicity of many of these substances are often unknown, and information about the long-term side effects of their use is scarce if not absent. Thus, there is a clear and unmet need to develop effective small-molecule proprotein convertase inhibitors that can better prevent or treat cardiovascular diseases that are facilitated by proprotein convertase activity.

### SUMMARY OF THE INVENTION

It is therefore an object of the invention to provide a therapeutically effective pharmaceutical enzyme inhibitor.

It is another object to provide a pharmaceutical composition which functions as a therapeutically effective pharmaceutical enzyme inhibitor.

It is a further object to include proprotein convertases as one compound within the pharmaceutical composition.

It is another object to provide furin and other related compounds as the proprotein convertases.

It is a further object to include one or more noble gases as another compound within the pharmaceutical composition.

These and other related objects are achieved according to a first embodiment of the invention by a pharmaceutical composition including two compounds. The first compound consists of one or more proprotein convertases; and the second compound consists of one or more isotopes of one or more noble gases, wherein the first and second compounds are combined to form a therapeutically effective pharmaceutical enzyme inhibitor. Gaseous oxygen may be combined with the second composition in a gaseous form. The gaseous oxygen is present in an amount of at least 20% by volume with the gaseous second composition present in an amount of about 0.5% to 79% by volume with the balance comprising proprotein convertases. Gaseous oxygen may be present in an amount up to 98.5%. The second compound is more specifically present in an amount of about 5% to 35% by volume, more particularly, about 5% to 25%.

The one or more proprotein convertases are selected from the group consisting of PCSK1, PCSK2, PCSK3, PCSK4, PCSK5, PCSK6, PCSK7, PCSK8, and PCSK9, any other proprotein convertase, and any analogue of these proprotein convertases in other species. The said one or more noble gases are selected from the group comprising helium, neon, argon, krypton, xenon, and radon. The said one or more proprotein convertases are selected from the group consisting of a human furin and a portion of a human furin. The said composition further includes one or more hydrophilic materials, and wherein said second compound is encapsulated in said one or more hydrophilic materials. The said one or more hydrophilic materials comprises cyclodextrin. The said one or more hydrophilic materials comprises (2-hydroxypropyl)-β-cyclodextrin (HPCD). The said compound encapsulated in hydrophilic material is present in about 0.5% to about 79% by weight within the composition. The said composition further includes one or more liposomes. The said second compound is dissolved in the one or more liposomes. The second composition dissolved in the one or more liposomes is present in about 0.5% to 79% by weight. The disclosure provides a composition, wherein said composition further includes gaseous oxygen combined with said second compound in a gaseous state. The combined gaseous oxygen and gaseous second compound includes at least 20% gaseous oxygen. The gaseous oxygen is present in a range of about between 20% and 94% by volume and the gaseous second composition is present in a range of about 5% to 79% by volume with the balance comprising proprotein convertases. The disclosure provides a composition wherein said composition further includes a respiratory stimulant. The respiratory stimulant is present in an amount between about 0.1% and 5% by volume. The respiratory stimulant is one of β2 adrenergic receptor agonists, doxapram, methylxanthines, esketamine and ampakines. The gaseous oxygen is present in a range of about between 20% and 94% by volume and the gaseous second composition is present in a range of about 5% to 79% by volume with the balance comprising proprotein convertases and the respiratory stimulant. The respiratory stimulant may be albuterol. The disclosure provides a composition wherein said one or more noble gases forms one or more compound(s) with one or more elements. The disclosure provides a composition wherein one or more enantiomers, or a racemic mixture thereof, of said compound formed by one or more noble gases, is present. The disclosure provides a composition wherein said pharmaceutical composition further comprises one or more pharmaceutically acceptable excipients.

These and other related objects are achieved according to a second embodiment of the invention providing a method of preventing and/or treating a cardiovascular disease, including the steps of: selecting a patient in need of preventing or treating a cardiovascular disease, and administering to the patient a pharmaceutical composition comprising one or more isotopes of one or more noble gases to form a therapeutically effective pharmaceutical enzyme inhibitor with the one or more proprotein convertases naturally present in the patient's body; whereby the cardiovascular disease is prevented or treated in the patient. The said one or more proprotein convertases are selected from the group consisting of PCSK1, PCSK2, PCSK3, PCSK4, PCSK5, PCSK6, PCSK7, PCSK8, and PCSK9, any other proprotein convertase, and any analogue of these proprotein convertases in other species. The said one or more noble gases are selected from the group comprising helium, neon, argon, krypton, xenon, and radon. The said one or more proprotein convertases are selected from the group consisting of a human furin and a portion of a human furin. The disclosure provides a method wherein said composition further includes one or more hydrophilic materials, and wherein said second compound is encapsulated in said one or more hydrophilic materials. The said one or more hydrophilic materials comprises cyclodextrin. The said one or more hydrophilic materials comprises (2-hydroxypropyl)-β-cyclodextrin (HPCD). The said composition further comprises one or more liposomes, and wherein said second compound is dissolved in said one or more liposomes. The said composition further includes gaseous oxygen combined with said second compound in a gaseous state. The combined gaseous oxygen and gaseous second compound includes at least 20% gaseous oxygen. The said composition further includes a respiratory stimulant. The said pharmaceutical composition further comprises a pharmaceutically acceptable excipient. The said composition may be delivered by any route of delivery. The said pharmaceutical composition is delivered orally, intravenously, by inhalation, by nebulization, by the intra-articular route, the intrathecal route, in any ophthalmic preparation, in any otic preparation; in any preparation for cutaneous delivery, including but not limited to a cream, lotion, ointment, gel, foam, or spray; by the intranasal route, by the suppository route, by the intravitreal route, and by intravitreal injection.

According to the method of preventing and/or treating a cardiovascular disease, the pharmaceutical composition includes two compounds. The first compound consists of one or more proprotein convertases; and the second compound consists of one or more isotopes of one or more noble gases, wherein the first and second compounds are combined to form a therapeutically effective pharmaceutical enzyme inhibitor. Gaseous oxygen may be combined with the second composition in a gaseous form. The gaseous oxygen is present in an amount of at least 20% by volume with the gaseous second composition present in an amount of about 0.5% to 79% by volume with the balance comprising proprotein convertases. Gaseous oxygen may be present in an amount up to 98.5%. The second compound is more specifically present in an amount of about 5% to 35% by volume, more particularly, about 5% to 25%. The one or more proprotein convertases are selected from the group consisting of PCSK1, PCSK2, PCSK3, PCSK4, PCSK5, PCSK6, PCSK7, PCSK8, and PCSK9, any other proprotein convertase, and any analogue of these proprotein convertases in other species. The one or more proprotein convertases are selected from the group consisting of a human furin and a portion of a human furin. The composition further includes a hydrophilic material. The second compound is encapsulated in the hydrophilic material. The second compound encapsulated in hydrophilic material is present in about 0.5% to about 79% by weight within the composition. The second compound encapsulated in hydrophilic material is more specifically present in an amount of about 5% to 35% by weight, more particularly, about 5% to 25%. The hydrophilic material is cyclodextrin or (2-hydroxypropyl)-β-cyclodextrin (HPCD). The composition further includes a liquid containing one or more liposomes. The second compound is dissolved in the liquid containing one or more liposomes. The second composition dissolved in the liquid containing one or more liposomes is present in about 0.5% to 79% by weight. The second compound dissolved in liquid is more specifically present in an amount of about 5% to 35% by weight, more particularly, about 5% to 25%. The composition further includes gaseous oxygen combined with the second compound in a gaseous state. The combined gaseous oxygen and gaseous second compound includes at least 20% gaseous oxygen. The gaseous oxygen is present in a range of about between 20% and 94% by volume and the gaseous second composition is present in a range of about 5% to 79% by volume with the balance comprising proprotein convertases. The composition further includes a respiratory stimulant. The respiratory stimulant is present in an amount between about 0.1% and 5% by volume. The respiratory stimulant is one of β2 adrenergic receptor agonists, doxapram, methylxanthines, esketamine and ampakines. The gaseous oxygen is present in a range of about between 20% and 94% by volume and the gaseous second composition is present in a range of about 5% to 79% by volume with the balance comprising proprotein convertases and the respiratory stimulant. The respiratory stimulant may be albuterol. The composition further comprises a pharmaceutically acceptable excipient. The said cardiovascular disease is selected from the group comprising pulmonary hypertension, hypertriglyceridemia, stable angina, unstable angina, atherosclerotic disease at any bodily site, coronary artery disease, atherosclerotic heart disease, peripheral vascular disease, vascular dementia, ischemic stroke, hemorrhagic stroke, acute mountain sickness, high altitude pulmonary edema, high altitude cerebral edema, flash pulmonary edema, neurogenic pulmonary edema, post-resuscitation pulmonary edema, type 2 diabetes mellitus, myocardial infarction, hypertensive heart disease, persistent pulmonary hypertension of the newborn, cardiomyopathy, dilated cardiomyopathy, restrictive cardiomyopathy, peripartum cardiomyopathy, and bronchopulmonary dysplasia.

According to a further embodiment, the invention provides a method of treating a cardiovascular disease in a patient comprising the steps of selecting a patient having a cardiovascular disease characterized by a TGF-beta signaling mechanism. A pharmaceutical composition is administered to the patient comprising a respiratory stimulant present in an amount between about 0.1% and 5% by volume, and a second compound comprising one or more naturally-occurring non-radioactive isotopes of one or more noble gases present in an amount between about 5% and 35% by volume. The one or more naturally-occurring non-radioactive isotopes of the one or more noble gases combine with a proprotein convertase comprising furin present in the patient's body to inhibit the proprotein convertase. The pharmaceutical composition is a therapeutically effective proprotein convertase inhibitor which decreases levels of TGF-beta production to treat the cardiovascular disease in the patient.

The cardiovascular disease is one of atherosclerosis, pulmonary hypertension, pulmonary arterial hypertension, hypertensive heart disease, cardiac fibrosis, ischemic stroke, diabetic cardiomyopathy, cardiomyopathy, dilated cardiomyopathy, ischemic cardiomyopathy, hypertrophic cardiomyopathy, valvular disease, arrhythmia and atrial fibrillation.

The administering step includes administering by one of inhalation or nebulization. The respiratory stimulant is selected from the group consisting of β2 adrenergic receptor agonists, doxapram, methylxanthines, esketamine and ampakines. In a further embodiment, the respiratory stimulant is albuterol. The pharmaceutical composition further includes gaseous oxygen combined with said second compound in a gaseous state. The combined gaseous oxygen and gaseous second compound includes at least 20% gaseous oxygen. In one embodiment the pharmaceutical composition includes the respiratory stimulant present in an amount between about 0.1% and 5% by volume, the noble gas present in an amount between about 5% and 35% by volume, and the balance an inert gas, oxygen or combinations thereof. The one or more noble gases are selected from the group consisting of helium, neon, argon, krypton and xenon.

The composition further includes one or more hydrophilic materials consisting of cyclodextrin or (2-hydroxypropyl)-β-cyclodextrin (HPCD), and wherein said second compound is encapsulated in said one or more hydrophilic materials. The hydrophilic material is cyclodextrin or (2-hydroxypropyl)-β-cyclodextrin (HPCD). The encapsulated second compound is administered by one of orally, intravenously, the intra-articular route, the intrathecal route, in an ophthalmic preparation, in an otic preparation, cutaneous delivery, by a cream, lotion, ointment, gel, foam, or spray, by the intranasal route, by the suppository route, by the intravitreal route, and by intravitreal injection.

In an alternate embodiment, the second compound is dissolved in a liquid containing one or more liposomes. The pharmaceutical composition further includes a pharmaceutically acceptable excipient. The second compound includes xenon. The second compound comprises a pharmaceutically-effective small-molecule proprotein convertase inhibitor. The level of furin activity is measured by an assay performed on a sample of a body fluid. The level of transforming growth factor-beta activity is measured by an assay performed on a sample of a body fluid.

### DETAILED DESCRIPTION OF THE PREFERRED EMBODIMENTS

Previous studies have suggested that medications that inhibit furin may improve the course of numerous cardiovascular diseases; a selected number of these are supported with references below.

One of the most important roles of our method of furin inhibition for cardiovascular disease is its role in treating atherosclerotic disease. Atherosclerosis is the process of producing cholesterol plaques in any vessel of the body; this process causes various diseases depending on where the plaques are located in the body, including but not limited to coronary artery disease, stable angina, and unstable angina (in the coronary arteries that provide the heart itself with blood), vascular dementia (in the brain), ischemic stroke (if a plaque breaks off and lodges in the brain), and peripheral vascular disease (atherosclerotic plaques in the vasculature of the extremities). Furin inhibition is a promising treatment for this disease process, as taught by Turpeinen et al: “Proprotein convertase subtilisin/kexin (PCSK) enzymes cleave proproteins into mature end products. Previously, MBTPS1 and PCSK9 have been shown to regulate cholesterol metabolism and LDL receptor recycling, whereas FURIN and PCSK5 have been suggested to inactivate lipases and regulate inflammation in atherosclerosis. Here, we systematically analyzed the expression of PCSKs and their targets in advanced atherosclerotic plaques. Microarray and quantitative real-time PCR experiments showed that FURIN (42.86 median fold, p=2.1e−8), but no other PCSK, is universally overexpressed in the plaques of different vascular regions . . . Immunohistochemistry experiments showed the upregulation of FURIN in the plaque lymphocytes and macrophages where it was co-expressed with BAFF/TNFSF13B and APRIL/TNFSF13. Our data unequivocally show that FURIN is the primary PCSK that is dysregulated in the immune cells of advanced human atherosclerotic plaques, which implies a role for this enzyme in plaque pathology. Therefore, drugs that inhibit FURIN in arteries may modulate the course of this disease.”

Another notable disease which may be improved by our method of furin inhibition is hypertriglyceridemia. Liu et al teach, “Lipoprotein lipase (LPL)-mediated lipolysis of triglycerides is the first and rate-limiting step in chylomicron/very low density lipoprotein clearance at the luminal surface of the capillaries. Angiopoietin-like protein 3 (ANGPTL3) is shown to inhibit LPL activity and plays important roles in modulating lipoprotein metabolism in vivo. However, the mechanism by which it inhibits LPL activity remains poorly understood. Using cell-based analysis of the interaction between ANGPTL3, furin, proprotein convertase subtilisin/kexin type 5 (PCSK5), paired amino acid converting enzyme-4 (PACE4), and LPL, we demonstrated that the cleavage of LPL by proprotein convertases is an inactivation process, similar to that seen for endothelial lipase cleavage. At physiological concentrations and in the presence of cells, ANGPTL3 is a potent inhibitor of LPL. This action is due to the fact that ANGPTL3 can enhance LPL cleavage by endogenous furin and PACE4 but not by PCSK5. This effect is specific to LPL but not endothelial lipase. Both N-and C-terminal domains of LPL are required for ANGPTL3-enhanced cleavage, and the N-terminal domain of ANGPTL3 is sufficient to exert its effect on LPL cleavage. Moreover, ANGPTL3 enhances LPL cleavage in the presence of either heparan sulfate proteoglycans or glycosylphosphatidylinositol-anchored high density lipoprotein-binding protein 1 (GPIHBP1). By enhancing LPL cleavage, ANGPTL3 dissociates LPL from the cell surface, inhibiting both the catalytic and noncatalytic functions of LPL. Taken together, our data provide a molecular connection between ANGPTL3, LPL, and proprotein convertases, which may represent a rapid signal communication among different metabolically active tissues to maintain energy homeostasis. These novel findings provide a new paradigm of specific protease-substrate interaction and further improve our knowledge of LPL biology.” By inhibiting furin, we can decrease the degradation of lipoprotein lipase, which increases its effectiveness in breaking down high levels of triglycerides in the blood. This would have important health benefits for many patients with unhealthful levels of serum triglycerides.

Of note, furin is important for the production of transforming growth factor-62 , which facilitates many different diseases. In the research study “Processing of transforming growth factor beta 1 precursor by human furin convertase”, Dubois et al report “Transforming growth factor (TGF)-betal plays an essential role in cell growth and differentiation. It is also considered as a gatekeeper of immune homeostasis with gene disruption leading to autoimmune and inflammatory diseases. TGF-betal is produced as an inactive precursor polypeptide that can be efficiently secreted but correct proteolytic cleavage is an essential step for its activation. Assessment of the cleavage site has revealed a unique R-H-R-R (SEQ ID NO:2) sequence reminiscent of proprotein convertase (PC) recognition motifs and has previously demonstrated that this PC-like cleavage site is correctly cleaved by furin, a member of the PC family. Here we report that among PC members, furin more closely satisfies the requirements needed to fulfill the role of a genuine TGF-betal convertase. Even though six members of the PC family have the ability to cleave TGF-betal, ectopic expression of alpha (1)-antitrypsin Portland (alpha(1)-AT-PDX), a potent furin inhibitor, blocked 80% of TGF-betal processing mediated by endogenous enzymes as demonstrated in an in vitro digestion assay. Genetic complementation of a furin-deficient LoVo cell line with the wild-type gene restores the production of mature and bioactivable TGF-beta1. Moreover, both furin and TGF-beta are coordinately expressed and regulated in vitro and in vivo in the hematopoietic and immune system, an important tissue target. These results demonstrate for the first time that furin is an authentic and adaptive TGF-beta1-converting enzyme whereas other members of the PC family might substitute or supplement furin activity. Our study advances our comprehension of the complexity of the TGF-beta system and should facilitate the development of therapeutically useful TGF-beta inhibitors.”

Since numerous genetic diseases are facilitated by TGF-β, a furin substrate, it is reasonable to expect that effective furin inhibitors would improve symptoms of these diseases by decreasing levels of TGF-β(in any of its isoforms) in the patient's body.

One cardiovascular disease associated with excess TGF-beta signaling that may be improved by our method of proprotein convertase inhibition is pulmonary hypertension. Guignabert and Humbert report, “Accumulating evidence supports the notion that a shift of the balance in favor of a TGF-β/activin/nodal signaling is occurring in human PAH, even in the absence of mutations in members of the TGF-βsuperfamily. This shift increases the risk to develop PAH and is strongly suspected to also contribute to disease pathogenesis by modulating cell survival, metabolism, inflammation, genome instability, migration and cell differentiation.”

Y. Zhang et al report similar findings in a rat model of pulmonary arterial hypertension (PAH): “Overexpressed TGF-β1 can activate the RhoA/ROCK signaling pathway, thus promoting the occurrence and development of PAH.”

Another similar opportunity lies in using our method in patients with hypertensive heart disease. Meng et al report, “Hypertension remains a major cause of chronic heart disease. Hypertensive cardiac remodeling, characterized by progressive cardiac fibrosis and inflammation associated with high blood pressure, is a major complication of hypertension. Angiotensin II (Ang II) has been regarded as a key mediator in hypertensive cardiac remodeling. Many studies have reported that Ang II mediates fibrosis directly and indirectly via transforming growth factor β (TGF-β1)/Smad3 signaling because Ang II can activate Smad3 directly via the AT1-p38/Extracellular signal-Eegulated Kinase (ERK) mitogen-activated protein kinase (MAPK)-Smad crosstalk pathway and indirectly by inducing TGF-β. Thus, activation of TGF-β/Smad signaling may be a central mechanism in the pathogenesis of hypertensive cardiac disease.”

Patients with ischemic stroke may also benefit from our method of proprotein convertase inhibition. Hemojuvelin, a furin substrate, has been implicated in the pathogenesis of post-ischemic stroke neuronal injury, as taught by Young et al: “Our study aimed to establish the role of hemojuvelin (HJV) in acute ischemic stroke (AIS). We performed immunohistochemistry for HJV expression in human brain tissues from 10 AIS and 2 non-stroke autopsy subjects. Plasma HJV was measured in 112 AIS patients within 48 h after stroke. The results showed significantly increased HJV expression in brain tissues from AIS patients compare to non-stroke subjects. After adjusting for clinical variables, plasma levels of HJV within 48 h after stroke were an independent predictor of poor functional outcome three months post-stroke (OR:1.78, 95% CI: 1.03-3.07; P=0.038). In basic part, Western blotting showed that HJV expression in mice brains was apparent at 3 h after middle cerebral artery occlusion (MCAO), and increased significantly at 72 h. In cultured cortical neurons, expression of HJV protein increased remarkably 24 h after oxygen glucose deprivation (OGD), and small interfering RNAs (siHJV) transfected OGD neurons had a lower apoptotic rate. Importantly, 72 h post-MCAO, HJV knockout mice had significantly smaller infarcts and less expression of cleaved caspase-3 protein compared with wild-type mice. In summary, HJV participates in the mechanisms of post-stroke neuronal injury, and that plasma HJV levels can be a potential early outcome indicator for AIS patients.”

Khan reports that recent studies indicate that dilated, ischemic and hypertrophic cardiomyopathies are all associated with raised levels of TGF-beta. In addition, Khan states, “In fact, the pathogenic effects of TGF-B have now been suggested to play a major role in valvular disease and arrhythmia, particularly atrial fibrillation.” These reports are supported by the study conducted by Teekakirikul.

The medical research discussed above demonstrates that a range of diseases are TGF-beta mediated, more particularly cardiovascular diseases. An exemplary listing of such diseases, includes atherosclerosis, pulmonary hypertension, pulmonary arterial hypertension, hypertensive heart disease, cardiac fibrosis, ischemic stroke, diabetic cardiomyopathy, cardiomyopathy, dilated cardiomyopathy, ischemic cardiomyopathy, hypertrophic cardiomyopathy, valvular disease, arrhythmia and atrial fibrillation.

Accordingly, it would be desirable to have a safe and pharmaceutically-effective treatment, for example, in the form of a small-molecule proprotein convertase inhibitor.

The invention proposes one or more naturally-occurring non-radioactive isotopes of noble gases administered with a respiratory stimulant, because the inventor has recently discovered that the noble gases are novel furin inhibitors. Since the furin enzyme converts transforming growth factor-beta (TGF-beta) from its inactive precursor to its active form, the naturally-occurring non-radioactive isotopes of noble gases act to decrease the production of TGF-beta and thereby treat diseases caused by excess TGF-beta activity.

The respiratory stimulant is selected from the following class of stimulants: β2 adrenergic receptor agonists, doxapram, methylxanthines, esketamine and ampakines. In one embodiment, the respiratory stimulant is albuterol, due to its familiarity among health care providers, its long safety record, its wide availability and low cost.

The noble gases include naturally-occurring non-radioactive isotopes of helium, neon, argon, krypton and xenon. In a further embodiment, the second composition includes one or more non-radioactive isotopes of xenon.

In an additional embodiment, the disease treated is one of pulmonary hypertension, pulmonary arterial hypertension, hypertensive heart disease and ischemic stroke.

Furthermore, our method may benefit patients with type 2 diabetes mellitus. This disease is associated with elevated TGF-beta levels, which may be a biomarker for diabetic neuropathy (as taught by Hussain et al), diabetic nephropathy (as taught by Mou et al and Qiao et al), and diabetic cardiomyopathy (as taught by Yue et al).

The invention relates to the noble gases as proprotein convertase inhibitors. The noble gases are a group of naturally occurring elements, comprising helium, neon, argon, krypton, xenon, and radon, that appear in the rightmost column of the periodic table of the elements. They occur at standard temperature and pressure as gases. Each noble gas naturally composes a small fraction of atmospheric air and may be collected from atmospheric air. The noble gases do not react chemically with other elements except in rare and extreme cases. For this reason, it is generally taught that noble gases are inert gases. They are non-flammable, non-explosive, and non-toxic (except that asphyxiation may occur if sufficient quantities of any noble gas displace oxygen and interfere with oxygen delivery to living creatures).

The noble gases have had two primary medical roles to date. In the United States, xenon-133 (which may be delivered in a xenon vehicle) is approved by the FDA for use in medical imaging procedures, including for imaging of the lungs and for evaluation of cerebral blood flow. The United States FDA has also approved xenon-127 for similar purposes. Neither of these roles require xenon or any of its isotopes to inhibit any proprotein convertase or any other enzyme.

In addition, some European jurisdictions have approved xenon for purposes of general anesthesia; it is thought to be superior to other commonly used inhaled anesthetics, but the increased cost of xenon has limited its use at present. It is thought that xenon's anesthetic effects is mediated by its antagonism of the NMDA receptor (a glutamate receptor found on neuron cells of the brain), rather than any of the proprotein convertases.

A helium-oxygen mixture known as heliox is sometimes used to assist in oxygen delivery to patients with a severe asthma attack who have not responded to conventional treatments. Heliox treatment relies on the lower density of the helium-oxygen mixture compared to regular air to facilitate the mixture's passage, with the oxygen it contains, through constrictions or obstructions in a patient's airway. This treatment also does not require helium to inhibit any enzyme.

Although there are other experimental clinical uses under investigation for the noble gases, these are the primary uses that have entered routine clinical practice in the United States. However, the functions disclosed herein do not seem to have been previously published by other investigators. Furthermore, there has not previously been any suggestion to combine the known benefits of proprotein convertases with the therapeutic benefits of one or more noble gases in a single pharmaceutical compound. Furthermore, there has not previously been any suggestion to administer a pharmaceutical composition to a patient containing both proprotein convertases and one or more noble gases.

These and other related objects are achieved according to a first embodiment of the invention by a pharmaceutical composition including two compounds. The first compound consists of one or more proprotein convertases; and the second compound consists of one or more isotopes of one or more noble gases, wherein the first and second compounds are combined to form a therapeutically effective pharmaceutical enzyme inhibitor. Gaseous oxygen may be combined with the second composition in a gaseous form. The gaseous oxygen is present in an amount of at least 20% by volume with the gaseous second composition present in an amount of about 0.5% to 79% by volume with the balance comprising proprotein convertases. Gaseous oxygen may be present in an amount up to 98.5%. The second compound is more specifically present in an amount of about 5% to 35% by volume, more particularly, about 5% to 25%.

The one or more proprotein convertases are selected from the group consisting of PCSK1, PCSK2, PCSK3, PCSK4, PCSK5, PCSK6, PCSK7, PCSK8, and PCSK9, any other proprotein convertase, and any analogue of these proprotein convertases in other species. The said one or more noble gases are selected from the group comprising helium, neon, argon, krypton, xenon, and radon. The said one or more proprotein convertases are selected from the group consisting of a human furin and a portion of a human furin. The said composition further includes one or more hydrophilic materials, and wherein said second compound is encapsulated in said one or more hydrophilic materials. The said one or more hydrophilic materials comprises cyclodextrin. The said one or more hydrophilic materials comprises (2-hydroxypropyl)-β-cyclodextrin (HPCD). The said compound encapsulated in hydrophilic material is present in about 0.5% to about 79% by weight within the composition. The said composition further includes one or more liposomes. The said second compound is dissolved in the one or more liposomes. The second composition dissolved in the one or more liposomes is present in about 0.5% to 79% by weight. The disclosure provides a composition, wherein said composition further includes gaseous oxygen combined with said second compound in a gaseous state. The combined gaseous oxygen and gaseous second compound includes at least 20% gaseous oxygen. The gaseous oxygen is present in a range of about between 20% and 94% by volume and the gaseous second composition is present in a range of about 5% to 79% by volume with the balance comprising proprotein convertases. The disclosure provides a composition wherein said composition further includes a respiratory stimulant. The respiratory stimulant is present in an amount between about 0.1% and 5% by volume. The respiratory stimulant is one of β2 adrenergic receptor agonists, doxapram, methylxanthines, esketamine and ampakines. The gaseous oxygen is present in a range of about between 20% and 94% by volume and the gaseous second composition is present in a range of about 5% to 79% by volume with the balance comprising proprotein convertases and the respiratory stimulant. The respiratory stimulant may be albuterol. The disclosure provides a composition wherein said one or more noble gases forms one or more compound(s) with one or more elements. The disclosure provides a composition wherein one or more enantiomers, or a racemic mixture thereof, of said compound formed by one or more noble gases, is present. The disclosure provides a composition wherein said pharmaceutical composition further comprises one or more pharmaceutically acceptable excipients.

These and other related objects are achieved according to a second embodiment of the invention providing a method of preventing and/or treating a cardiovascular disease, including the steps of: selecting a patient in need of preventing or treating a cardiovascular disease, and administering to the patient a pharmaceutical composition comprising one or more isotopes of one or more noble gases to form a therapeutically effective pharmaceutical enzyme inhibitor with the one or more proprotein convertases naturally present in the patient's body; whereby the cardiovascular disease is prevented or treated in the patient. The said one or more proprotein convertases are selected from the group consisting of PCSK1, PCSK2, PCSK3, PCSK4, PCSK5, PCSK6, PCSK7, PCSK8, and PCSK9, any other proprotein convertase, and any analogue of these proprotein convertases in other species. The said one or more noble gases are selected from the group comprising helium, neon, argon, krypton, xenon, and radon. The said one or more proprotein convertases are selected from the group consisting of a human furin and a portion of a human furin. The disclosure provides a method wherein said composition further includes one or more hydrophilic materials, and wherein said second compound is encapsulated in said one or more hydrophilic materials. The said one or more hydrophilic materials comprises cyclodextrin. The said one or more hydrophilic materials comprises (2-hydroxypropyl)-β-cyclodextrin (HPCD). The said composition further comprises one or more liposomes, and wherein said second compound is dissolved in said one or more liposomes. The said composition further includes gaseous oxygen combined with said second compound in a gaseous state. The combined gaseous oxygen and gaseous second compound includes at least 20% gaseous oxygen. The said composition further includes a respiratory stimulant. The said pharmaceutical composition further comprises a pharmaceutically acceptable excipient. The said composition may be delivered by any route of delivery. The said pharmaceutical composition is delivered orally, intravenously, by inhalation, by nebulization, by the intra-articular route, the intrathecal route, in any ophthalmic preparation, in any otic preparation; in any preparation for cutaneous delivery, including but not limited to a cream, lotion, ointment, gel, foam, or spray; by the intranasal route, by the suppository route, by the intravitreal route, and by intravitreal injection.

According to the method of preventing and/or treating a cardiovascular disease, the pharmaceutical composition includes two compounds. The first compound consists of one or more proprotein convertases; and the second compound consists of one or more isotopes of one or more noble gases, wherein the first and second compounds are combined to form a therapeutically effective pharmaceutical enzyme inhibitor. Gaseous oxygen may be combined with the second composition in a gaseous form. The gaseous oxygen is present in an amount of at least 20% by volume with the gaseous second composition present in an amount of about 0.5% to 79% by volume with the balance comprising proprotein convertases. Gaseous oxygen may be present in an amount up to 98.5%. The second compound is more specifically present in an amount of about 5% to 35% by volume, more particularly, about 5% to 25%. The one or more proprotein convertases are selected from the group consisting of PCSK1, PCSK2, PCSK3, PCSK4, PCSK5, PCSK6, PCSK7, PCSK8, and PCSK9, any other proprotein convertase, and any analogue of these proprotein convertases in other species. The one or more proprotein convertases are selected from the group consisting of a human furin and a portion of a human furin. The composition further includes a hydrophilic material. The second compound is encapsulated in the hydrophilic material. The second compound encapsulated in hydrophilic material is present in about 0.5% to about 79% by weight within the composition. The second compound encapsulated in hydrophilic material is more specifically present in an amount of about 5% to 35% by weight, more particularly, about 5% to 25%. The hydrophilic material is cyclodextrin or (2-hydroxypropyl)-β- cyclodextrin (HPCD). The composition further includes a liquid containing one or more liposomes. The second compound is dissolved in the liquid containing one or more liposomes. The second composition dissolved in the liquid containing one or more liposomes is present in about 0.5% to 79% by weight. The second compound dissolved in liquid is more specifically present in an amount of about 5% to 35% by weight, more particularly, about 5% to 25%. The composition further includes gaseous oxygen combined with the second compound in a gaseous state. The combined gaseous oxygen and gaseous second compound includes at least 20% gaseous oxygen. The gaseous oxygen is present in a range of about between 20% and 94% by volume and the gaseous second composition is present in a range of about 5% to 79% by volume with the balance comprising proprotein convertases. The composition further includes a respiratory stimulant. The respiratory stimulant is present in an amount between about 0.1% and 5% by volume. The respiratory stimulant is one of β2 adrenergic receptor agonists, doxapram, methylxanthines, esketamine and ampakines. The gaseous oxygen is present in a range of about between 20% and 94% by volume and the gaseous second composition is present in a range of about 5% to 79% by volume with the balance comprising proprotein convertases and the respiratory stimulant. The respiratory stimulant may be albuterol. The composition further comprises a pharmaceutically acceptable excipient. The said cardiovascular disease is selected from the group comprising pulmonary hypertension, hypertriglyceridemia, stable angina, unstable angina, atherosclerotic disease at any bodily site, coronary artery disease, atherosclerotic heart disease, peripheral vascular disease, vascular dementia, ischemic stroke, hemorrhagic stroke, acute mountain sickness, high altitude pulmonary edema, high altitude cerebral edema, flash pulmonary edema, neurogenic pulmonary edema, post-resuscitation pulmonary edema, type 2 diabetes mellitus, myocardial infarction, hypertensive heart disease, persistent pulmonary hypertension of the newborn, cardiomyopathy, dilated cardiomyopathy, restrictive cardiomyopathy, peripartum cardiomyopathy, and bronchopulmonary dysplasia.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

### REFERENCES

Throughout this application, various references describe the state of the art to which this invention pertains. The contents of these documents are hereby incorporated into the present application by reference thereto.
DUBOIS CM et al, Evidence that furin is an authentic transforming growth factor-beta 1-converting enzyme. Am J Pathol. 2001 January;158(1):305-16: pubmed.ncbi.nlm.nih.gov/11141505/: 10.1016/s0002-9440(10)63970-3.
TURPEINEN H et al, Proprotein convertases in human atherosclerotic plaques: the overexpression of FURIN and its substrate cytokines BAFF and APRIL. Atherosclerosis. 2011 December; 219(2):799-806, pubmed.ncbi.nlm.nih.gov/21889147/: 10.1016/j.atherosclerosis.2011.08.011.
ZHANG Y et al, TGF-β1 promotes pulmonary arterial hypertension in rats via activating RhoA/ROCK signaling pathway. Eur Rev Med Pharmacol Sci. 2020 May;24(9):4988-4996, pubmed.ncbi.nlm.nih.gov/32432762/: 10.26355/eurrev_202005_21190.
LIU J et al, Angiopoietin-like protein 3 inhibits lipoprotein lipase activity through enhancing its cleavage by proprotein convertases. J Biol Chem. 2010 Sep. 3;285(36):27561-70, pubmed.ncbi.nlm.nih.gov/20581395/: 10.1074/jbc.M110.144279.
YOUNG GH et al, The functional role of hemojuvelin in acute ischemic stroke. J Cereb Blood Flow Metab. 2020 June;40(6):1316-1327, pubmed.ncbi.nlm.nih.gov/31307288/: 10.1177/0271678X19861448.
HUSSAIN G et al, Serum levels of TGF-β1 in patients of diabetic peripheral neuropathy and its correlation with nerve conduction velocity in type 2 diabetes mellitus. Diabetes Metab Syndr. 2016 January-March;10(1 Suppl 1):S135-9, pubmed.ncbi.nlm.nih.gov/26559756/: 10.1016/j.dsx.2015.10.011.
MOU X et al, Serum TGF-β1 as a Biomarker for Type 2 Diabetic Nephropathy: A Meta-Analysis of Randomized Controlled Trials. PLoS One. 2016 Feb. 22;11(2):e0149513, pubmed.ncbi.nlm.nih.gov/26901047/: 10.1371/journal.pone.0149513.
QIAO YC et al, Changes of transforming growth factor beta 1 in patients with type 2 diabetes and diabetic nephropathy: A PRISMA-compliant systematic review and meta-analysis. Medicine (Baltimore). 2017 April;96(15):e6583, pubmed.ncbi.nlm.nih.gov/28403088/: 10.1097/MD.0000000000006583.
LIANG D et al, Metformin inhibits TGF-beta 1-induced MCP-1 expression through BAMBI-mediated suppression of MEK/ERK½ signalling. Nephrology (Carlton). 2019 April;24(4):481-488, pubmed.ncbi.nlm.nih.gov/29934960/: 10.1111/nep.13430.
SUN XJ et al, Effects of aerobic exercise and resveratrol on the expressions of JAK2 and TGF-β1 in renal tissue of type 2 diabetes rats. Zhongguo Ying Yong Sheng Li Xue Za Zhi. 2020 May;36(3):202-206. Chinese: 10.12047/j.cjap.5918.2020.045.
YUE Y et al, Transforming growth factor beta (TGF-β) mediates cardiac fibrosis and induces diabetic cardiomyopathy. Diabetes Res Clin Pract. 2017 November;133:124-130, pubmed.ncbi.nlm.nih.gov/28934669/: 10.1016/j.diabres.2017.08.018.
DOBACZEWSKI M et al, Transforming growth factor (TGF)-β signaling in cardiac remodeling. J Mol Cell Cardiol. 2011 October;51(4):600-6. doi: 10.1016/j.yjmcc.2010.10.033. Epub 2010 Nov. 6, pubmed.ncbi.nlm.nih.gov/21059352/.
KHAN R et al, Fibrosis in heart disease: understanding the role of transforming growth factor-beta in cardiomyopathy, valvular disease and arrhythmia. Immunology. 2006 May;118(1):10-24, pubmed.ncbi.nlm.nih.gov/16630019/: 10.1111/j.1365-2567.2006.02336.x.
DENG S et al, Transforming Growth Factor-β-Neutralizing Antibodies Improve Alveolarization in the Oxygen-Exposed Newborn Mouse Lung. J Interferon Cytokine Res. 2019 February;39(2):106-116, pubmed.ncbi.nlm.nih.gov/30657417/: 10.1089/jir.2018.0080.
ZHAO et al, Adenovirus-mediated decorin gene transfer prevents TGF-beta-induced inhibition of lung morphogenesis. Am J Physiol. 1999 August;277(2): L412-22, pubmed.ncbi.nlm.nih.gov/10444536/: 10.1152/ajplung.1999.277.2.L412.
SAKURAI R et al, Curcumin augments lung maturation, preventing neonatal lung injury by inhibiting TGF-β signaling. Am J Physiol Lung Cell Mol Physiol. 2011 November;301(5):L721-30, pubmed.ncbi.nlm.nih.gov/21821729/10.1152/ajplung.00076.2011.
GUIGNABERT C et al, Targeting transforming growth factor-β receptors in pulmonary hypertension. Eur Respir J. 2021 Feb. 4;57(2):2002341, pubmed.ncbi.nlm.nih.gov/32817256/: 10.1183/13993003.02341-2020.
MENG J et al, Treatment of Hypertensive Heart Disease by Targeting Smad3 Signaling in Mice. Mol Ther Methods Clin Dev. 2020 Aug. 5;18:791-802, pubmed.ncbi.nlm.nih.gov/32953930/: 10.1016/j.omtm.2020.08.003.

What is claimed is:

1. A method of treating a cardiovascular disease in a patient comprising the steps of:
selecting a patient having a cardiovascular disease characterized by a TGF-beta signaling mechanism; and
administering to the patient a pharmaceutical composition comprising a respiratory stimulant present in an amount between about 0.1% and 5% by volume, and a second compound comprising one or more naturally-occurring non-radioactive isotopes of one or more noble gases present in an amount between about 5% and 35% by volume, wherein the one or more naturally-occurring non-radioactive isotopes of the one or more noble gases combine with a proprotein convertase comprising furin present in the patient's body to inhibit the proprotein convertase;
wherein the pharmaceutical composition is a therapeutically effective proprotein convertase inhibitor which decreases levels of TGF-beta production to treat the cardiovascular disease in the patient.

2. The method of claim 1, wherein the cardiovascular disease is one of atherosclerosis, pulmonary hypertension, pulmonary arterial hypertension, hypertensive heart disease, cardiac fibrosis, ischemic stroke, diabetic cardiomyopathy, cardiomyopathy, dilated cardiomyopathy, ischemic cardiomyopathy, hypertrophic cardiomyopathy, valvular disease, arrhythmia and atrial fibrillation.

3. The method of claim 2, wherein the administering step includes administering by one of inhalation or nebulization.

4. The method of claim 1, wherein the administering step includes administering by one of inhalation or nebulization.

5. The method of claim 3, wherein said respiratory stimulant is selected from the group consisting of β2 adrenergic receptor agonists, doxapram, methylxanthines, esketamine and ampakines.

6. The method of claim 3, wherein said respiratory stimulant is albuterol.

7. The method of claim 1, wherein said respiratory stimulant is albuterol.

8. The method of claim 1, wherein said pharmaceutical composition further includes gaseous oxygen combined with said second compound in a gaseous state.

9. The method of claim 8, wherein the combined gaseous oxygen and gaseous second compound includes at least 20% gaseous oxygen.

10. The method of claim 3, wherein said one or more noble gases are selected from the group consisting of helium, neon, argon, krypton and xenon.

11. The method of claim 1, wherein said one or more noble gases are selected from the group consisting of helium, neon, argon, krypton and xenon.

12. The method of claim 1, wherein said composition further includes one or more hydrophilic materials consisting of cyclodextrin or (2-hydroxypropyl)-β-cyclodextrin (HPCD), and wherein said second compound is encapsulated in said one or more hydrophilic materials.

13. The method of claim 12, wherein said one or more hydrophilic materials is cyclodextrin.

14. The method of claim 12, wherein said one or more hydrophilic materials is (2-hydroxypropyl)-β-cyclodextrin (HPCD).

15. The method of claim 12, wherein the encapsulated second compound is administered by one of orally, intravenously, the intra-articular route, the intrathecal route, in an ophthalmic preparation, in an otic preparation, cutaneous delivery, by a cream, lotion, ointment, gel, foam, or spray, by the intranasal route, by the suppository route, by the intravitreal route, and by intravitreal injection.

16. The method of claim 1, wherein said second compound is dissolved in a liquid containing one or more liposomes.

17. The method of claim 1, wherein said pharmaceutical composition further comprises a pharmaceutically acceptable excipient.

18. The method of claim 1, wherein the second compound includes xenon.

19. The method of claim 1, wherein the second compound comprises a pharmaceutically-effective small-molecule proprotein convertase inhibitor.

20. The method of claim 1, where the level of furin activity is measured by an assay performed on a sample of a body fluid.

21. The method of claim 1, wherein the level of transforming growth factor-beta activity is measured by an assay performed on a sample of a body fluid.
